(19) United States
(12) Patent Application Publication
(10) Pub. No.: US 2019/0159779 A1
(43) Pub. Date: May 30, 2019

(54) METHOD OF PERFORMING AN OSTOMY PROCEDURE

(71) Applicant: Covidien LP, Mansfield, MA (US)

(72) Inventor: AVVLN Srinivasa Murthy Aravalli, Tanuku (IN)

(21) Appl. No.: 16/202,441

(22) Filed: Nov. 28, 2018

Related U.S. Application Data
(60) Provisional application No. 62/591,699, filed on Nov. 28, 2017.

(51) Int. Cl. A61B 17/11; A61B 17/115; A61B 17/34

(52) U.S. Cl. CPC A61B 17/1114; A61F 5/445; A61B 17/3415; A61B 17/1155

(57) ABSTRACT

A method of performing an ostomy procedure includes forming a first incision in an abdominal wall of a patient; inserting a cannula into the first incision into an abdominal cavity of the patient; forming a second incision in the abdominal wall of the patient; inserting a first shell assembly of a circular stapling device into the second incision; delivering an anvil assembly of the circular stapling device through the cannula into the abdominal cavity; securing the anvil assembly to an anvil retainer of the circular stapling device; approximating the anvil assembly and the shell assembly to clamp a rectus sheath of the patient between the anvil assembly and the shell assembly; firing the circular stapling device to form a circular opening in the rectus sheath, the circular opening being reinforced by an annular array of staples.

### CROSS-REFERENCE TO RELATED APPLICATIONS

This application claims the benefit of and priority to U.S. Provisional Patent Application No. 62/591,699 filed Nov. 28, 2017, the entire disclosure of which is incorporated by reference herein.

### BACKGROUND

### 1. Technical Description

The present disclosure is directed to a method of performing an ostomy procedure and, more particularly, to a method of performing an ostomy procedure to avoid complications such as parastomal herniation.

### 2. Background of Related Art

Exteriorization of an internal body vessel such as the intestine is called a stoma. Stomas may be created in conjunction with an ostomy procedure, e.g., colostomy or ileostomy, by suturing a bisected portion of an intestine to the abdominal wall to provide internal access into the intestine for collecting fecal matter. Parastomal herniation is the most significant and frequent complication associated with patient's that have had ostomy procedures. Parastomal herniation generally refers to a bulge or swelling around the stoma that leads to problems with stoma function.

Parastomal herniation may result when the abdominal muscles, e.g., the rectus sheath, separate from the stoma. This separation usually occurs gradually and may worsen over time and may allow an internal body organ to enter the space between the internal body organ, generally a section of the bowel, and the abdominal muscles, which may result in strangulation of the internal body organ.

A continuing need exists in the art for an improved procedure for forming stoma that minimizes the likelihood of parastomal herniation from occurring.

### SUMMARY

One aspect of the disclosure is directed to a method of performing an ostomy procedure including forming a first incision in an abdominal wall of a patient; inserting a cannula into the first incision into an abdominal cavity of the patient; forming a second incision in the abdominal wall of the patient; inserting a first shell assembly of a circular stapling device into the second incision; delivering an anvil assembly of the circular stapling device through the cannula into the abdominal cavity; securing the anvil assembly to an anvil retainer of the circular stapling device; approximating the anvil assembly and the shell assembly to clamp a rectus sheath of the patient between the anvil assembly and the shell assembly; and firing the circular stapling device to form a circular opening reinforced by an annular array of staples in the rectus sheath.

In some embodiments, the method includes separating the anvil assembly from the anvil retainer and removing the anvil assembly from the abdominal cavity.

In certain embodiments, removing the anvil assembly from the abdominal cavity is conducted through the cannula.

In embodiments, removing the anvil assembly from the abdominal cavity is conducted through the second incision.

In some embodiments, the method further includes securing a portion of a bowel of a patient to the abdominal wall to form a stoma.

In certain embodiments, securing a portion of the bowel to the abdominal wall includes securing the portion of the bowel to a dermis of the patient.

In embodiments, securing the portion of the bowel of the patient to the abdominal wall is performed with a second circular stapling device.

In some embodiments, the method further includes removing the second stapling device from a patient through the stoma.

### BRIEF DESCRIPTION OF THE DRAWINGS

Various embodiments of the presently disclosed method of performing an ostomy procedure are described herein below with reference to the drawings, wherein:

FIG. 1 is a side perspective view of an abdominal area of a patient with a cannula extending through a first incision in an abdominal wall into the abdominal cavity and a circular stapling device extending through a second incision in the abdominal wall into the abdominal cavity;

FIG. 2 is a side cross-sectional view of the abdominal cavity shown in FIG. 1 with an anvil assembly of the circular stapling device positioned adjacent to an anvil retainer of the circular stapling device prior to attachment of the anvil assembly to the anvil retainer;

FIG. 3 is a side cross-sectional view of the abdominal cavity and the circular stapling device shown in FIG. 2 with the anvil assembly of the circular stapling device attached to the anvil retainer of the circular stapling device and positioned on an internal side of the rectus sheath and a shell assembly of the circular stapling device positioned on an opposite side of the rectus sheath prior to approximation of the anvil assembly and the shell assembly;

FIG. 4 is a side cross-sectional view of the abdominal cavity and circular stapling device shown in FIG. 2 with the anvil assembly of the circular stapling device attached to the anvil retainer of the circular stapling device and positioned on the internal side of the rectus sheath and the shell assembly of the circular stapling device positioned on the opposite side of the rectus sheath with the anvil assembly and the shell assembly approximated about the rectus sheath prior to firing of the circular stapling device;

FIG. 5 is a side cross-sectional view of the abdominal cavity and circular stapling device shown in FIG. 2 with the anvil assembly attached to the anvil retainer of the circular stapling device and positioned on the internal side of the rectus sheath and the shell assembly of the circular stapling device positioned on the opposite side of the rectus sheath with the anvil assembly and the shell assembly approximated about the rectus sheath after the circular stapling device has been fired;

FIG. 5A is a side cross-sectional view of the abdominal cavity and circular stapling device shown in FIG. 2 with the anvil assembly attached to the anvil retainer of the circular stapling device and positioned on the internal side of the cutaneous tissue of the abdominal wall with the cutaneous tissue clamped between the anvil assembly and the shell assembly of the circular stapling device;

FIG. 6 is a top view of a stoma stapled to the dermis of the abdominal wall; and

FIG. 7 is a side view of the stoma and the abdominal wall illustrating the body organ in phantom.

### DETAILED DESCRIPTION OF EMBODIMENTS

The presently disclosed method of performing an ostomy procedure will now be described in detail with reference to the drawings in which like reference numerals designate identical or corresponding elements in each of the several views. However, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “clinician” is used generally to refer to medical personnel including doctors, nurses, and support personnel.

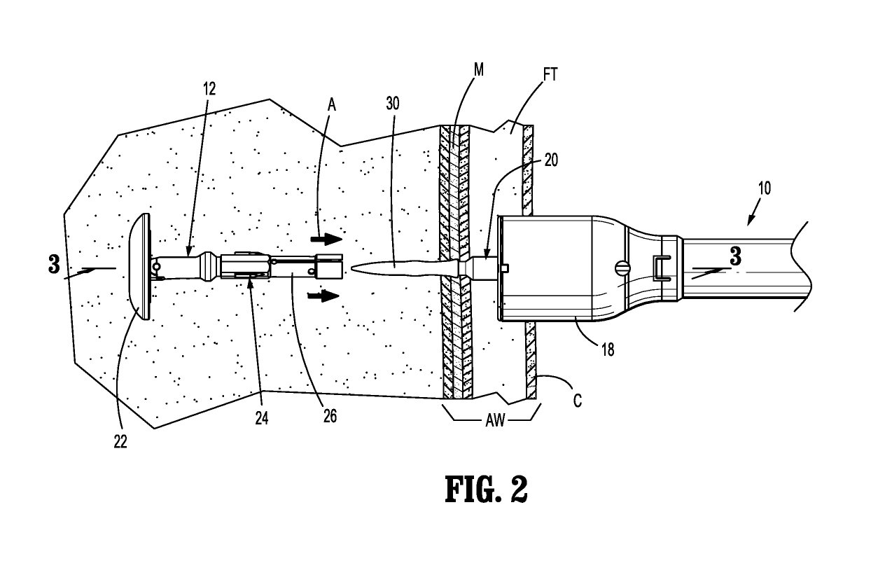

During an ostomy procedure, e.g., colostomy or ileostomy, a surgically created opening is formed in the abdominal wall for the discharge of body waste. As used herein, the abdominal wall “AW” includes an outer cutaneous layer “c” (e.g., epidermis, dermis, and hypodermis), a layer of fatty tissue “FT”, and an inner muscle or tissue layer “M” (e.g., anterior rectus sheath) that enshroud organs, vessels, and/or other tissue for performing various bodily functions such as digestion (FIG. 2). A portion of the bowel, e.g., colon or intestine, is secured to the abdominal wall to deliver body waste to the surgically created opening. The portion of the colon or intestine that is visible protruding through the surgically created opening is called a stoma. The most significant and frequent complication associated with patient's that have had ostomy procedures is parastomal herniation. Parastomal herniation generally refers to a bulge or swelling around the stoma that leads to problems with stoma function. A parastomal hernia can occur when edges of the stoma separate from the abdominal muscles such that abdominal contents, generally a portion of the bowel, bulge through the space defined between the stoma and the abdominal muscles. The presently disclosed method of performing an ostomy procedure provides a reinforced opening through tissue defining the abdominal wall to minimize the likelihood of parastomal herniation.

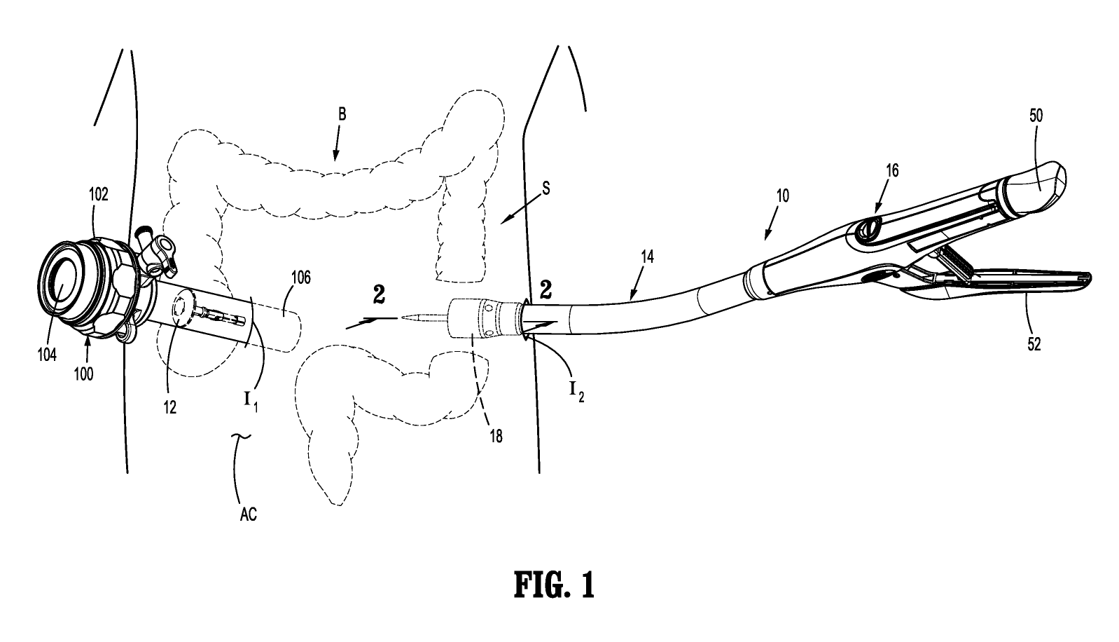

An exemplary embodiment of the presently disclosed method of performing an ostomy procedure is shown in FIGS. 1-7. Referring to FIG. 1, in the presently disclosed method of performing an ostomy procedure, a circular stapling device 10 is used to provide a reinforced opening “O” (FIG. 7) in a muscle layer “M” of the abdominal wall “AW” and to secure a portion of the bowel “B” to the cutaneous layer “c” of the abdominal wall to form a stoma “ST” (FIG. 7). The circular stapling device 10 includes an anvil assembly 12, a body portion 14, a shell assembly 18 supported on a distal portion of the body portion 14, and a handle assembly 16 supported on a proximal end of the body portion 14. Although not described in detail herein, the circular stapling device 10 can be as described in any one of U.S. Pat. Nos. 9,307,994 (the '994 Patent), 9,532,781 and 6,957,758 and U.S. Patent Publication No. 2012/0253329 which are incorporated herein in their entirety by reference. Alternately, other known circular stapling devices can be used to perform the presently described procedure.

Referring to FIG. 1, after a procedure in which a portion of bowel “B” is removed and prior to performing an anastomosis of the separated bowel portions, a circular stapling device 10 is delivered to the surgical site “S” within the abdominal cavity “AC”. More specifically, in the presently disclosed method, the anvil assembly 12 of the circular stapling device 10 is delivered to the abdominal cavity “AC” through a cannula assembly 100. As known in the art, the cannula assembly 100 includes a port 102 defining a sealed opening 104 and a cannula body 106 that extends distally from the sealed opening 104. The cannula body 106 is positioned through a first incision “I1” in the abdominal wall “AW”. The anvil assembly 102 is inserted through the sealed opening 104 and advanced into the abdominal cavity “AC” through the cannula body 106. The shell assembly 18 of the circular stapling device 10 is positioned through a second incision “I2” formed in the abdominal wall “AW” at a location to position the stoma “ST” (FIG. 7).

Referring to FIG. 2, after the anvil assembly 12 and the shell assembly 18 are properly positioned, the anvil assembly 12 is attached to an anvil retainer 20 of the circular stapling device 10. In embodiments, the anvil assembly 12 includes an anvil head 22, and a hollow shaft 24 that is partially defined by flexible fingers 26, and the anvil retainer 20 includes a trocar 30. The trocar 30 is received within the hollow shaft 24 in the direction indicated by arrow “A” such that the flexible fingers 26 releasably engage the trocar 30. See, e.g., the '994 Patent.

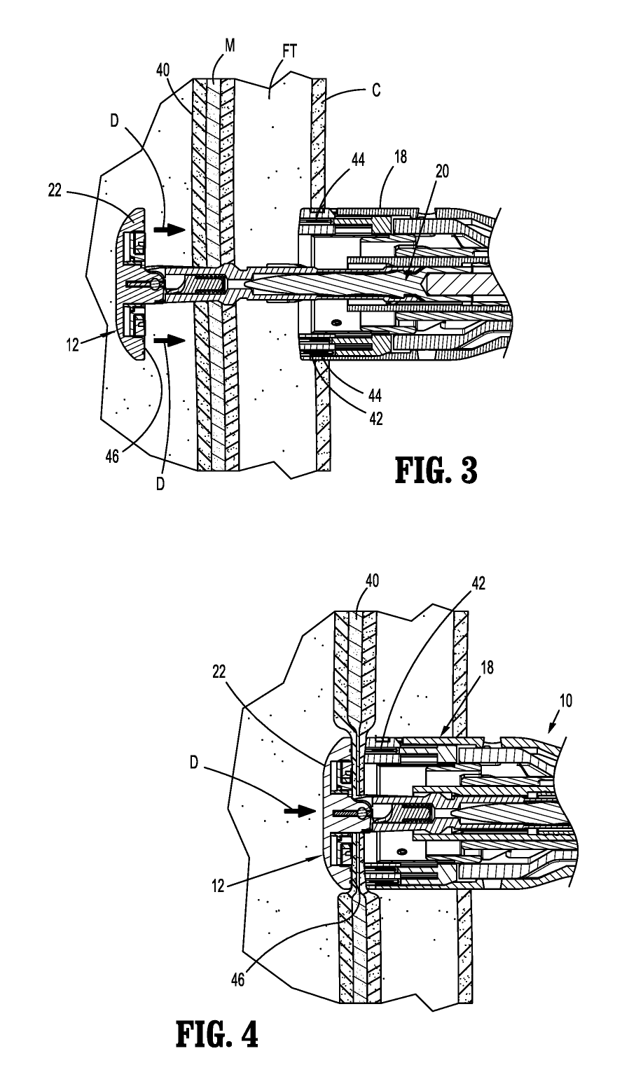

Referring to FIG. 3, after the anvil assembly 12 is secured to the anvil retainer 20, the anvil head 22 is positioned internally of the rectus sheath 40 and the shell assembly 18 is positioned externally of the muscle layer “M” of the abdominal wall “AW”, e.g., the rectus sheath 40, such that the rectus sheath 40 is positioned between the anvil head 22 and the shell assembly 18. The shell assembly 18 includes a staple cartridge 42 that supports a plurality of staples 44 and the anvil head 22 has a staple deforming surface 46. The rectus sheath 40 is positioned between the staple deforming surface 46 of the anvil head 22 and staple cartridge 42 of the shell assembly 18.

Referring also to FIG. 4, after the circular stapling device 10 is properly positioned about the rectus sheath 40, the anvil assembly 12 is approximated towards the staple cartridge 42 in the direction indicated by arrows “D” by actuating an approximations knob 50 (FIG. 1) of the circular stapling device 10 to clamp the rectus sheath 40 between the staple deforming surface 46 of the anvil head 22 of the anvil assembly 12 and the staple cartridge 42 of the shell assembly 18. For a detailed description of the operation of a circular stapling device such as stapling device 10, see the '994Patent.

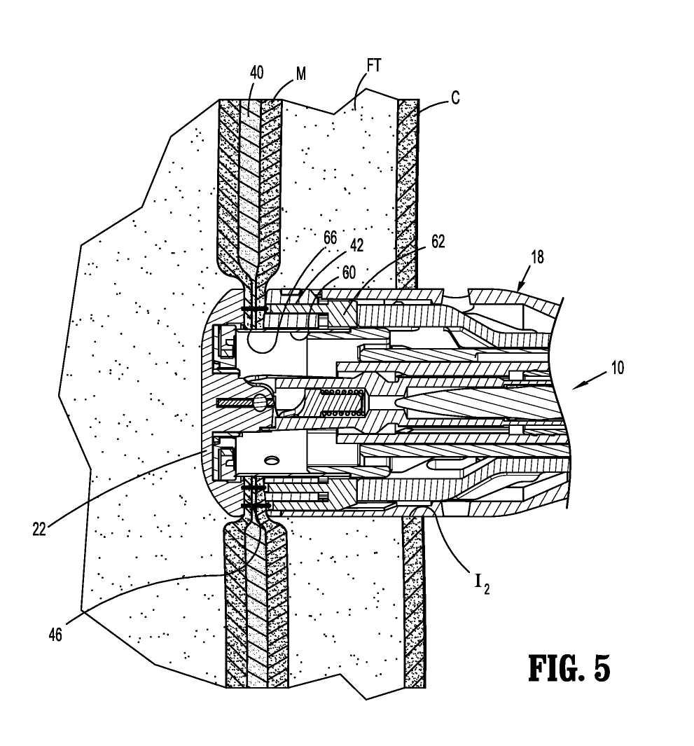

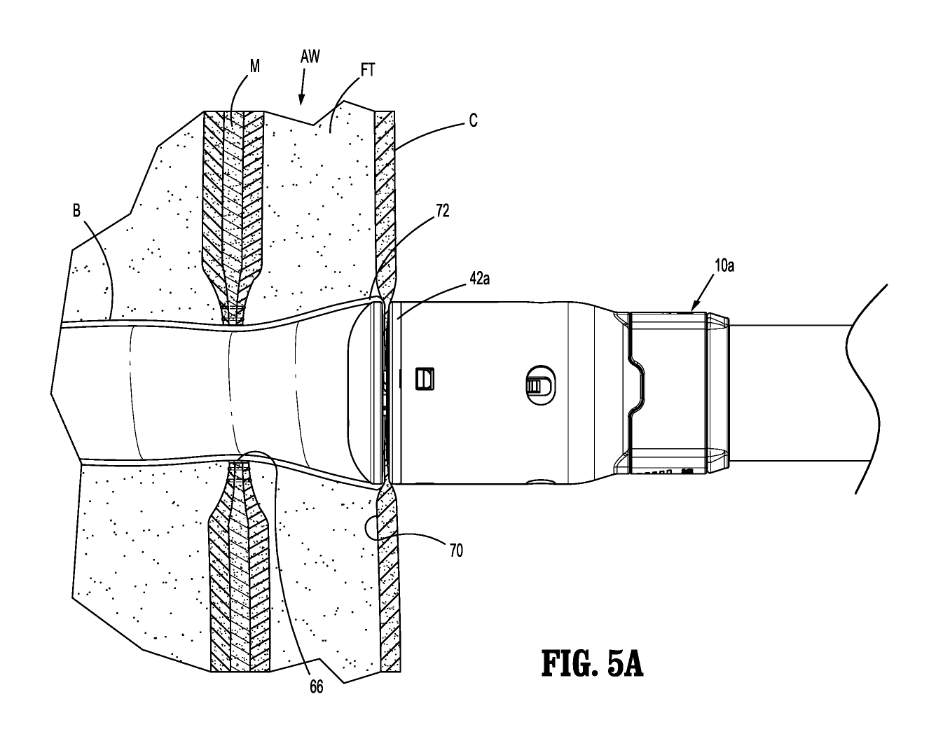

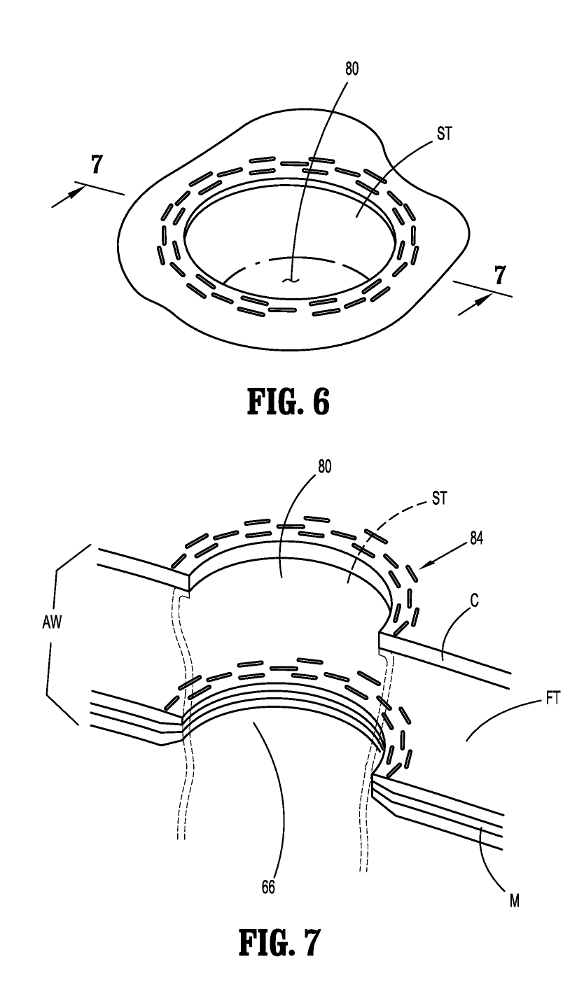

Referring to FIGS. 5 and 6, after the rectus sheath 40 is properly positioned between the staple deforming surface 46 of the anvil head 22 and staple cartridge 42 of the shell assembly 18, the circular stapling device 10 is fired by compressing the firing trigger 52 (FIG. 1) of the circular stapling device 10. The shell assembly 18 includes an annular knife 60 and a pusher 62. When the circular stapling device 10 is fired, the annular knife 60 and the pusher 62 are advanced towards the anvil assembly 12 to form a circular opening 66 in the rectus sheath 40 and provide a circular array of the staples 44 about the opening 66 to reinforce the opening 66. After the opening 66 is formed in the rectus sheath 40, the anvil assembly 12 can be separated from the anvil retainer 20 of the circular stapling device 10 and removed from the abdominal cavity through the cannula 100 (FIG. 1). The shell assembly 18 can then be removed from the patient through the second incision 12. Alternately, the anvil assembly 12 can be removed from the abdominal cavity through the circular opening 66 and the second incision 12 (FIG. 7).

Referring to FIGS. 5A-7, after the circular stapling device 10 is removed from the abdominal cavity, the portion of the bowel “B” to be exteriorized is withdrawn through the reinforced opening 66 in the muscle layer “M” of the abdominal wall “AW”. Next, a new or second circular stapling device 10A is used to secure the portion of the bowel “B” to be exteriorized to the dermis 70 of the cutaneous layer “c” of the abdominal wall “AW” to form a stoma 72. As used herein, “a second circular stapling device 10A” includes stapling device 10 with a new shell assembly 18A including a fresh staple cartridge or, alternatively, a different circular stapling device 10 with a fresh staple cartridge 42A. In embodiments, the circular stapling device 10A is positioned to compress the end of the bowel “B” to the dermis 70 of the cutaneous layer “c” of the abdominal wall “AW”. When the circular stapling device 10 is fired, a circular opening 80 (FIG. 7) that is reinforced by an annular array of staples 84 is created in the abdominal wall “AW” and the portion of the bowel “B” to be exteriorized is secured to the dermis 70 to form the stoma “ST”. After the stoma “ST” is formed, the anvil assembly 12 can be separated from the anvil retainer 20 and removed from the patient through the stoma 72.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A method of performing an ostomy procedure comprising:
forming a first incision in an abdominal wall of a patient;
inserting a cannula into the first incision into an abdominal cavity;
forming a second incision in the abdominal wall of the patient;
inserting a first shell assembly of a circular stapling device into the second incision;
delivering an anvil assembly of the circular stapling device through the cannula into the abdominal cavity;
securing the anvil assembly to an anvil retainer of the circular stapling device;
approximating the anvil assembly and the shell assembly to clamp a rectus sheath of the patient between the anvil assembly and the shell assembly; and
firing the circular stapling device to form a circular opening that is reinforced by an annular array of staples in the rectus sheath.

2. The method of claim 3, further including separating the anvil assembly from the anvil retainer and removing the anvil assembly from the abdominal cavity.

3. The method of claim 2, wherein removing the anvil assembly from the abdominal wall is conducted through the cannula.

4. The method of claim 3, wherein removing the anvil assembly from the abdominal cavity is conducted through the second incision.

5. The method of claim 1, further including securing a portion of a bowel of a patient to the abdominal wall to form a stoma.

6. The method of claim 2, wherein securing a portion of the bowel to the abdominal wall includes securing the portion of the bowel to a dermis of the abdominal wall of the patient.

7. The method of claim 4, wherein securing the portion of the bowel of the patient to the abdominal wall is performed with a second circular stapling device.

8. The method of claim 3, further including removing the second stapling device from the stoma.
